(19) United States
(12) Patent Application Publication
(10) Pub. No.: US 2003/0140925 A1
(43) Pub. Date: Jul. 31, 2003

(54) System for conditioning expiratory muscles for an improved respiratory system

(76) Inventors: Christine A. Sapienza, Gainesville, FL (US); Anatole D. Martin, Gainesville, FL (US); Paul Wesley Davenport, Gainesville, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/349,426

(22) Filed: Jan. 22, 2003

Related U.S. Application Data
(63) Continuation-in-part of application No. 09/908,329, filed on Jul. 18, 2001, now Pat. No. 6,568,387.
(60) Provisional application No. 60/219,307, filed on Jul. 19, 2000.
(60) Provisional application No. 60/351,256, filed on Jan. 22, 2002.

(51) Int. Cl. A61M 16/00

(52) U.S. Cl. 128/205.24; 128/204.18; 128/204.23

(57) ABSTRACT

The present invention is directed to breathing methods and devices, which increase intra-airway pressure, thus causing a positive expiratory pressure (PEP) which is not airflow dependent. Specifically, in a preferred embodiment, the present invention provides methods that utilize a pressure relief valve, preferably a positive end-expiratory pressure (PEEP) valve, for providing positive expiratory pressure (PEP). The PEP is caused by directing the flow of gases exhaled by the patient through the PEEP valve, so that gases must be exhaled against the PEEP valve held closed by threshold pressure. In this way, gases exhaled by the patient are subject to positive exhalation pressure set by the threshold pressure, which in turn increase the pressure in the patient's airway. When the expiratory pressure exceeds the threshold pressure of the valve, the valve opens and air is exhaled. The present invention is further directed to a unique training system, which uses the PEEP valve to increase respiratory muscle strength. More specifically, the present invention provides methods to increase the expiratory airflow rate and endurance of the expiratory muscles in a person by increasing the ability of these muscles to force air out of the lungs.

### CROSS-REFERENCE TO RELATED APPLICATIONS

This application claims the benefit of U.S. Provisional application No. 60/351,256, filed Jan. 22, 2002. This application is also a continuation-in-part of U.S. application Ser. No. 09/908,329, filed Jul. 18, 2001, which claims the benefit of U.S. Provisional application Serial No. 60/219,307, filed Jul. 19, 2000.

### FIELD OF INVENTION

The present invention relates generally to the field of breathing exercise and more particularly to an expiratory breathing method which promotes proper pressure breathing by the user. The present invention further generally relates to systems for providing unique physical training programs designed to increase respiratory muscle strength for an improved respiratory system.

### BACKGROUND OF INFORMATION

The process of inspiration refers to the use of the diaphragm and chest muscles to inflate the lung and breathe air into the lung. The process of expiration refers to when a person lets air out of the lung, which typically does not involve the use of the respiratory muscles. When a person is engaged in an activity which necessitates strenuous expiration, such as speaking and singing, he or she recruits the muscles of the abdomen and chest to give a pumping force for expiration. The movement of air out of the lung (expiration) requires a positive pressure driving force which is greater in the alveoli than at the mouth. This creates a pressure gradient down which air will move by bulk flow.

There are two sources of positive pressure during expiration: 1) the recoil pressure generated by the elasticity of the lung, and 2) active compression of the lung with contraction of the expiratory muscles. The lung has an elasticity that is measured as compliance. An analogy is a balloon. When the balloon is inflated, the latex is stretched from its rest position. Holding the end of an inflated balloon closed one can feel the positive pressure inside the balloon as the latex squeezes the air in the balloon attempting to return to its rest position. If the balloon end is opened, air will flow out of the balloon because of the pressure gradient generated by the elastic recoil of the balloon wall.

The lung has elasticity and when the lung is inflated with a large inspiration, the lung walls are stretched. The elastic lung tissue compresses the air in the lung creating a positive pressure that is proportional to the lung stretch, the lung volume. Active contraction of the expiratory muscles squeezes the outer surface of the lung adding to the positive pressure by further compressing the air in the lung. Again, this is analogous to putting an inflated balloon in your hands and squeezing the balloon.

For example, as shown in FIG. 1, the net positive pressure in the lung is the sum of the elastic recoil pressure and the expiratory muscle squeeze pressure. Thus, if the elastic recoil pressure with an inflated lung is 10 cmH₂O and the expiratory muscles squeeze the lung with 30 cmH₂O, the total positive pressure in the alveoli is 40 cmH₂O. These pressures are referenced to atmospheric pressure which we consider 0 cmH₂O (i.e., the alveolar pressure is 40 cmH₂O greater than atmospheric pressure). It is also important to recognize that the pressure in the alveoli is 40 cmH₂O and the pressure at the mouth (or nose) is atmospheric or 0 cmH₂O. This means that the pressure decreases along the airways going from the alveoli to the mouth with all 40 cmH₂O dissipating along this path. The pressure is lost due to the resistance of the respiratory tract.

Another important feature of the respiratory anatomy is that the lung and all the airways are within the thorax except for approximately half the trachea, the pharynx, and mouth. This means that when the expiratory muscles contract, the squeeze pressure is applied to the entire thoracic cavity, which applies the squeeze pressure equally to the entire lung (alveoli and airways) within the thorax. In our example, that means that 30 cmH₂O squeezing pressure is applied to the alveoli and the intrathoracic airways. The alveoli have a net positive pressure of 40 cmH₂O because of the combination of the elastic recoil pressure and the expiratory muscle squeeze pressure. This results in a greater pressure in the alveoli than outside the alveoli and the alveoli stay distended. As noted above, however, the intra-airway pressure decreases due to loss of pressure from airway resistance. That means the closer to the mouth, the lower the positive pressure inside the airway. At some point in this path, the intra-airway pressure will decrease to 30 cmH₂O. This happens in intrathoracic airways. At this point, the pressure inside the airway equals the expiratory muscle squeeze pressure outside the airway and is called the Equal Pressure Point (EPP).

Moving closer to the mouth from the EPP results in a further decrease in the intra-airway pressure. Now, the intra-airway pressure is less than the expiratory muscle squeeze pressure and there is a net collapsing force applied to the airway. As the airway is compressed, the resistance increases and more pressure is lost due to the elevated resistive forces.

The reason peak expiratory airflow during forced expirations is effort-independent is because the greater the expiratory effort, the greater the expiratory muscle squeeze, and the greater the compression force beyond the EPP. This increased airway compression increases the resistance and dissipates more pressure as air flows through the compressed airway. This creates a physical limit to the maximum airflow because no matter how much greater the positive pressure from active expiratory muscle contraction driving force, there is a proportional increase in airway collapse, limiting the airflow, making the peak airflow rate measured at the mouth effort-independent.

In the normal lung, the EPP occurs in bronchi that contain cartilage. The cartilage limits the compression of the airway and protects the airway from collapse with forced expirations. With emphysema, as shown in FIG. 2, there is a loss of lung elasticity recoil, meaning that with inflation of the lung the elastic recoil pressure portion of the positive alveolar pressure is decreased. When the expiratory muscles contract during emphysema, as in the example above, a 30 cmH₂O squeeze pressure is generated. The net alveolar pressure is now 30 cmH₂O squeeze pressure with a reduced elastic recoil pressure, for example 5 cmH₂O, making the net alveolar pressure 35 cmH₂O. Again, pressure is dissipated as air flows towards the mouth. With this emphysema example, the EPP will occur closer to the alveoli as the intra-airway pressure goes from 35 to 30 cmH₂O quicker than the normal lung which went from 40 to 30 cmH₂O. Thus, the EPP moves closer to the alveoli and can even occur in bronchioles which are airways that do not have cartilage.

If the EPP occurs in non-cartilaginous airways, as in emphysema, then airway collapse can occur due to the expiratory muscle squeeze pressure being greater than the intra-airway pressure with no cartilage to prevent the collapse of the airway. When the airway collapses, gas is trapped in the lung and the patient cannot fully empty their lungs resulting in hyperinflation, called dynamic hyperinflation. In this condition, exhaling with a greater effort provides no relief because dynamic airway compression (and/or collapse) causes forced expiratory efforts to be effort independent. Dynamic airway collapse during expiration is a major problem in patients with chronic obstructive airways (pulmonary) disease (COPD).

One method used to assist patients with COPD, including emphysema patients with this type of gas trapping, is to use pursed-lips breathing. This requires patients to breathe out through their mouths with the lips partially closed as if they were whistling. This increases the airflow resistance at the mouth creating an elevated pressure behind the lip obstruction, like partially covering a water hose with your thumb which creates a higher pressure behind the obstruction. Pursed-lips breathing increases the pressure down the respiratory tract creating a positive expiratory pressure (PEP) and functionally moves the EPP closer to the mouth. This is an airflow-dependent (because it works only when air is moving) method of compensating for dynamic airway collapse in COPD patients. This prevents some of the collapse of the airways and permits additional deflation of the lung, reducing the dynamic hyperinflation.

Increasing the intra-airway pressure during expiration by creating a positive expiratory pressure (PEP) is an important method for maintaining airway patency, decreasing gas trapping and reducing hyperinflation in emphysema patients. Several attempts have been made to manufacture resistance devices which imitate pursed-lips breathing, including U.S. Pat. Nos. 4,523,137 to Sonne; 4,601,465 to Ray; and 5,598,839 to Niles. These devices are successful in producing the same effect as pursed-lips breathing, but only marginally effective in reducing the dynamic hyperinflation in severe COPD cases. The marginal effectiveness in reducing the dynamic hyperinflation in severe COPD cases occurs as a result of the method being airflow-dependent, i.e., there is no PEP unless the patient is actually moving air. Thus, when airflow is maximized, the PEP effect is maximum, and the EPP will be moved closer to the mouth. However, most COPD patients cannot generate and sustain high expiratory airflows. In fact, the expiratory airflow pattern is characterized by the peak airflow early in the expiration with a rapidly diminished airflow. As the expiration progresses, airflow tails-off, with very low flows as the expiration ends. This results in very little PEP in the latter half of the expiration which abolishes much of the effect of dynamic hyperinflation reduction.

Most COPD patients live a restricted lifestyle because of severe breathlessness, inability to exercise and need for supplemental oxygen. When queried, most patients are desperate for a solution to reduce their primary distressing symptom, breathlessness. Clinicians need non-pharmacological methods to improve the O₂ and CO₂ status of the patient and to treat the dynamic hyperinflation. Current use of bronchodilators and resistance breathing methods are helpful but produce only modest improvements in many cases. What is needed is a significant PEP throughout the entire expiration which will keep the airways open allowing them to properly deflate. This would then decrease end-expiratory lung volume, allow for better inspiratory pumping (by making the diaphragm go closer to its optimal contraction length), increase alveolar ventilation, increase the O₂ in the blood, decrease the CO₂ in the blood, and decrease the sense of breathlessness that causes great distress in COPD patients.

COPD patients, however, are not the only ones who suffer from loss of intra-airway pressure caused by airway resistance. Another disorder related to expiratory airflow is vocal fold disorder, which creates high upper airway resistance in patients with the disorder. One of the causes of this disorder is the result of scarring of the vocal folds from a variety of origins. In addition, vocal fold paralysis produces upper airway resistance, or vocal fold edema, which results from overuse of the vocal folds. For patients with these vocal fold disorders, training of their expiratory muscles to increase the muscles' strength to force air out of the lungs provides some treatment. Strengthening the expiratory muscles provides greater expiratory muscle pressure and reduces the stress on the vocal folds, resulting in less laryngeal compensation to the vocal folds.

While strengthening the expiratory muscles helps the patients with vocal fold disorder by increasing the muscle pressure, such improvement of the muscle power also provides certain benefits to a normal, healthy person's respiratory system. For example, when a person is engaged in an activity which necessitates strenuous expiration, such as speaking, singing or playing a wind instrument, he or she recruits the muscles of the abdomen and chest to give a pumping force for expiration. A training program to increase the strength of these expiratory muscles to better force air out of the lungs would increase the expiratory airflow rate (increasing the magnitude of the sound) and endurance (ability to produce a strong force longer). In other words, training expiratory muscles to become stronger can assist people to increase the sound intensity of their voice or to generate efficient blowing pressure for playing instruments. Many speakers and musicians perform exercises to increase their sound or voice strength or blow pressure, often requiring hours of training with limited success.

Patients with obstructive sleep apnea syndrome (OSAS) have upper airway obstruction or narrowing during sleep as well as impaired ability to compensate for upper airway narrowing. During sleep, respiratory muscle activity is decreased, ventilation is slowed and upper airway muscles have a reduced tone. This can lead to collapse of the upper airway with breathing efforts during sleep. The control of upper airway tone and ventilation is via the respiratory central neural motor drive system activating the inspiratory and expiratory muscle motor neurons.

Currently, there are no breathing device-based medical and/or therapeutic methods in use to condition the respiratory system and/or increase expiratory muscle strength. The conventional breathing exercises used by speech pathologists, vocal coaches and otolaryngologists produce only modest increases in maximum expiratory pressure (MEP) but require long training periods. What is needed is a system which not only treats patients with respiratory disorders, such as COPD, by methods of increasing alveolar ventilation, but which also significantly increases the expiratory pressure to make the respiratory muscles work near their maximum force-generating capacity. Specifically, what is needed is a simple and more effective breathing method, with short training periods, which enables a user to increase the strength of the expiratory muscles, which also in turn provides medical treatments to patients with vocal fold-related expiratory resistance disorders by decreasing the stress on their vocal folds.

### BRIEF SUMMARY OF THE INVENTION

The present invention is directed to breathing methods and devices which increase intra-airway pressure, thus causing a positive expiratory pressure (PEP) which is not airflow dependent. Specifically, in a preferred embodiment, the present invention provides methods which utilize a pressure relief valve, preferably a positive end-expiratory pressure (PEEP) valve, for providing positive expiratory pressure (PEP). The PEP is caused by directing the flow of gases exhaled by the patient through the PEEP valve, so that gases must be exhaled against the PEEP valve held closed by threshold pressure. In this way, gases exhaled by the patient are subject to positive exhalation pressure set by the threshold pressure, which in turn increase the pressure in the patient's airway. When the expiratory pressure exceeds the threshold pressure of the valve, the valve opens and air is exhaled.

The present invention is further directed to a unique training system which uses the PEEP valve to increase respiratory muscle strength. More specifically, the present invention provides methods to increase the expiratory airflow rate and endurance of the expiratory muscles in a person by increasing the ability of these muscles to force air out of the lungs.

In accordance with the practice of the present invention, patients breathe out through a PEEP valve, generating enough pressure to overcome the PEEP valve's pressure threshold, allowing air to flow through the PEEP valve. Expiring through the PEEP valve creates a PEP equal to the PEEP valve's pressure. The PEP produced by the PEEP valve results in increased airway patency, such that the amount of gas trapped in the lung decreases (reduced hyperinflation) and the airway resistance decreases.

The elevated PEP from a PEEP valve remains in the airway throughout the expiration, even to the very end, moving the Equal Pressure Point (EPP) closer to the mouth, and keeping it there. The decreased hyperinflation returns the diaphragm closer to its normal length, increasing the ability of the diaphragm to generate the inspiratory pumping forces. Improving the ability of these patients to ventilate their lungs increases their exercise tolerance and decreases their sense of breathlessness.

One aspect of the present invention is a unique PEEP valve which has been modified by changing the spring within the valve and/or changing the mouthpiece. With these modifications, the valve is now particularly advantageous for treating patients with COPD problems, or for aiding those who wish to strengthen expiration muscles and, with the implementation of a series of exercises to be used by individual people with vocal chord problems.

The novel application of the PEEP valve according to the present invention provides an inexpensive and non-pharmacological method of reducing breathlessness, increasing exercise capacity and improving alveolar ventilation. The novel application of the PEEP valve, and the accompanying exercise program, according to the present invention further provides a short and simple respiratory muscle training method for enhancing the respiratory system, and strengthening of expiratory muscles for increased sound intensity and decreased stress on vocal folds.

In one embodiment of the subject invention, exercises are provided that specifically strengthen the muscles of inspiration and expiration in order to increase the ability of patients to maintain a patent airway during sleep thus reducing the number and severity of respiratory obstructions during sleep. Exercises that increase respiratory muscle strength can also be used to increase upper pharyngeal tone during sleep and prevent airway closure, thereby improving breathing during sleep.

All patents, patent applications and publications referred to or cited herein, are incorporated by reference in their entirety to the extent they are not inconsistent with the explicit teachings of this specification.

### BRIEF DESCRIPTION OF THE FIGURES

FIG. 1 depicts an example of a patient's health lung exhalation pressure.

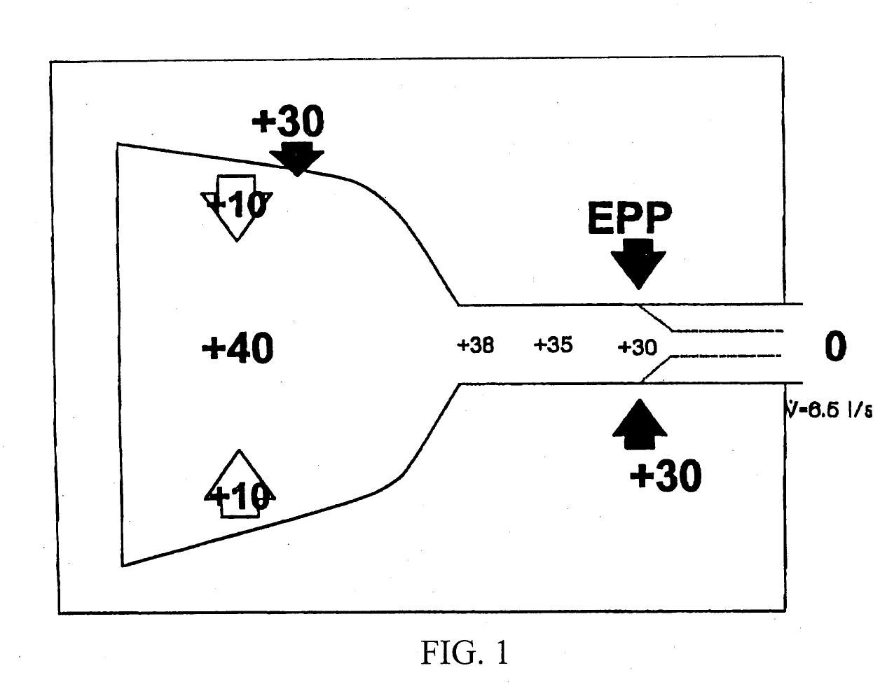

FIG. 2 depicts an example of exhalation pressure for a patient with emphysema.

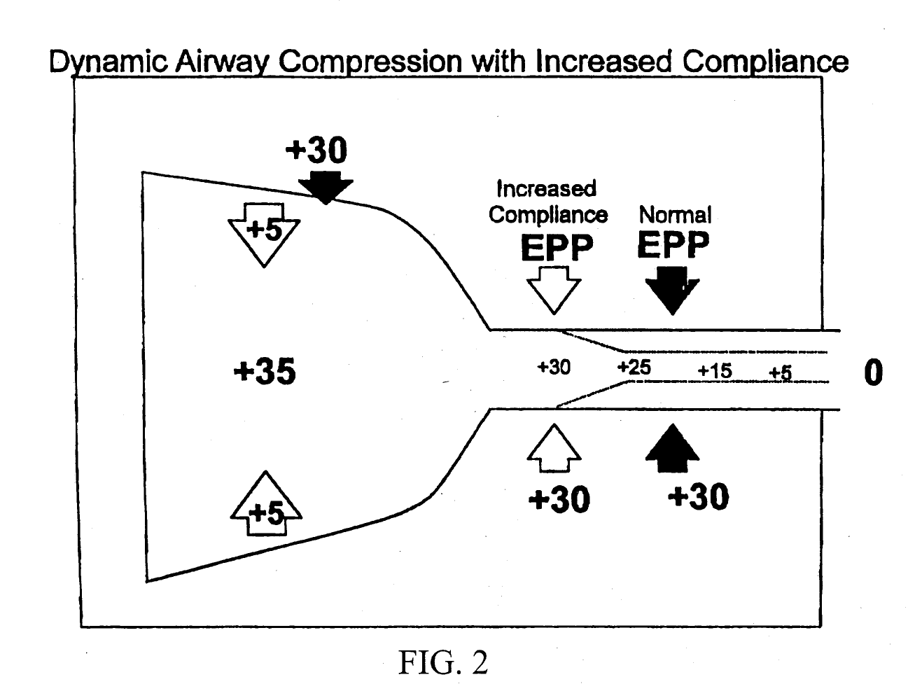

FIG. 3 shows an example of a PEEP valve.

FIG. 4 depicts a diagram of a modified device of the subject invention based on the Threshold Trainer.

FIG. 5 depicts a diagram of a modified PEEP valve device of the subject invention.

### DETAILED DESCRIPTION OF THE INVENTION

The subject invention provides a method which increases intra-airway pressure in a person, thus causing a positive expiratory pressure (PEP) which is not airflow dependent. The method of the present invention utilizes a pressure relief valve, preferably a positive end-expiratory pressure (PEEP) valve. The PEP is caused by obstructing the flow of gases exhaled by the patient through the PEEP valve until the PEP is greater than the pressure threshold of the PEEP valve, so that such gases must be exhaled against the PEEP valve's pressure threshold. Based on the same mechanism, the subject invention also provides a method to condition respiratory muscles by utilizing the PEEP valve to generate an increased expiratory force, which is not dependent on airflow or breathing rate, for strengthening expiratory muscles.

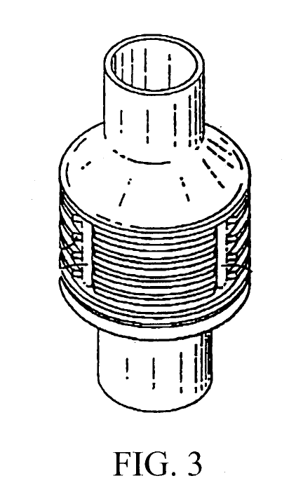

Examples of PEEP valves include, but are not limited to, U.S. Pat. No. 5,878,743 to Zdrojkowski, as shown in FIG. 3, which discloses an unidirectional valve with a spring force to control exhalation pressure; U.S. Pat. No. 1,896,719, to McKesson, which discloses a mask having an exhaling valve with a spring force adjustable by a set screw to control exhalation pressure; and U.S. Pat. No. 4,182,366, to Boehringer, which discloses a spring connected to a diaphragm, whereby the spring urges the diaphragm to close the exhaust port. A thumb screw can be adjusted to control the pressure on the spring; U.S. Pat. No. 4,207,884, to Isaacson, discloses an annular seat on a disk-shaped valve, whereby a spring urges the valve against its seat in accordance with the setting on a graduated plunger. Additional PEEP valves are disclosed in U.S. Pat. Nos. 4,403,616, 4,345,593, 4,870,963, and 5,109,840.

In accordance with the practice of the subject invention, the COPD patient breathes out through a valve, generating sufficient pressure to overcome the valve's pressure threshold, allowing air to flow through the valve. Expiring through the valve creates a PEP equal to the valve's pressure threshold. The PEP produced by the valve results in increased airway patency, such that the amount of gas trapped in the lung decreases (reduced hyperinflation) and the airway resistance decreases. The elevated PEP from the valve, equal to the pressure threshold of the valve, remains in the airway throughout the expiration. This decreases end-expiratory lung volume, allows for better inspiratory pumping, increases alveolar ventilation, increases the O₂ in the blood, decreases the CO₂ in the blood, and decreases the sense of breathlessness that causes great distress in these patients. Also, the decreased hyperinflation returns the diaphragm closer to its normal length, increasing the ability of the diaphragm to generate the inspiratory pumping forces. This improves the ability of the patient to ventilate the lungs, increasing exercise tolerance, and decreasing the sense of breathlessness.

OSAS patients have a reduced (compared to non-OSAS subjects) resting tone of the upper airway during sleep. This reduction in upper airway tone makes these patients more susceptible to upper airway collapse and obstruction of the glottis during sleep, hence obstructive sleep apnea. High intensity, short duration respiratory muscle strength training in accordance with the subject invention elicits a central respiratory motor pattern change that results in increased upper airway tone in OSAS patients thus reducing the tendency of the upper airway to collapse during sleep.

In one embodiment, the subject invention provides respiratory (inspiratory and expiratory) muscle strength training (RMST) to increase respiratory muscle strength and increase upper airway muscle compensation for upper airway narrowing during sleep. Thus, the materials and methods of the subject invention can be used to strengthen the muscles of inspiration and expiration to increase the motor capacity of the respiratory muscles and increase the ability of OSAS patients to maintain a patent airway during sleep, thus reducing the number and severity of respiratory obstructions during sleep. This can also help reduce snoring.

In another embodiment of the subject invention, a valve is utilized to increase intra-airway pressure in a patient, thus causing a positive expiratory pressure (PEP) and functionally moving the Equal Pressure Point (EPP) closer to the mouth. The valve is aligned such that the valve's threshold pressure resists the patients exhalation, whereby the threshold pressure is at a level such that the patient is capable of overcoming it upon exhalation through the valve. Initially, the patient inhales, filling the lungs, and then exhales through the valve with sufficient force to overcome the valve's threshold pressure. This inhalation and exhalation is referred to as a breathing cycle.

In accordance with one embodiment of the subject invention, the valve comprises a mouth piece, which is placed in the patient's mouth.

In one embodiment, the method of the subject invention is performed while the patient is at rest, or at limited activity. The valve threshold pressure is set to a relatively low threshold pressure level, about 1-5 cmH₂O. The patient continually exhales through the valve for a short duration of time, about 2-5 breaths, or about 0.05-1.5 minutes. The method is performed on a regular basis, with the valve threshold pressure being increased as the patient's tolerance increases.

In the “at-rest” embodiment, to increase the pressure in the patient's intra-airway the valve's threshold pressure is set to about 1-50 cm H₂O. In one specific at-rest embodiment, the valve threshold pressure is set to about 10 cmH₂O.

In an alternative at-rest embodiment, the duration of continual valve usage is increased as the patient's tolerance increases. To increase the pressure in a patient's intra-airway, the patient breathes through the valve continually for about 2-30 breaths. In one embodiment, the patient continually breathes through the valve for about 0.05 to about 30 minutes.

In an alternative method of use, the patient utilizes the valve while performing physical exercise, such as cardiovascular training. The valve threshold pressure is set to a low threshold pressure level, about 1-5 cmH₂O. While exercising, the patient continually exhales through the valve. Initially, the patient will exercise for a relatively short duration, about 0.05-5 min. As the patient's tolerance increases, the duration of the exercise increases.

In the “increased activity” embodiment, to increase the pressure in the patient's intra-airway, the pressure threshold of the valve is set to about 1-50 cm H₂O. In a specific increased activity embodiment, the valve threshold pressure is set to about 10 cmH₂O. The duration of continual exercise may be, for example, for about 0.05 to 30 minutes.

In accordance with yet another embodiment of the subject invention, a device of the subject invention is utilized in conjunction with daily breathing exercise sessions in order to condition the respiratory muscles. In one embodiment, the device exhibits substantial modification and improvement over the existing conventional, spring-loaded threshold pressure valves, such as positive end-expiratory pressure (PEEP) valves and the Threshold Trainer (manufactured by Respironics, Inc.), in producing the expiratory pressure threshold load. In a preferred embodiment, this newly improved device is equipped with increased spring constants which have, in one embodiment, a maximum valve opening pressure of about 160 cmH₂O, which is eight times higher than those provided by conventional threshold valves. The conventional threshold valves are equipped with a spring having spring constant that results in a maximum valve opening pressure (greatest spring compression) level of only 20 cmH₂, which is too low of a pressure to produce any significant expiratory muscle strengthening to subjects with normal respiratory muscles.

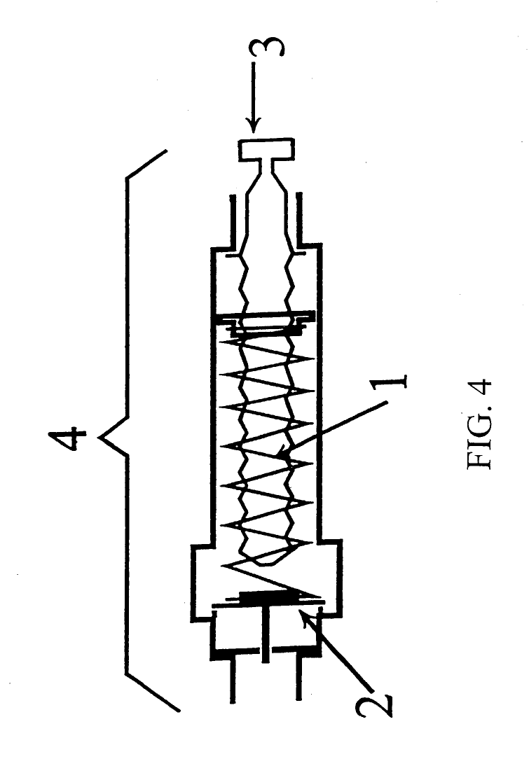

Now referring to FIG. 4, a modified device 4 of the subject invention based on the Threshold Trainer is depicted. The spring constant 1 embodied by the existing Threshold Trainer is too weak, with a threshold pressure range of 0-40, to produce respiratory muscle training for normal human subjects. Therefore, the spring constant 1 has been modified and improved in the device of the subject invention to result in a threshold pressure ranging from 0 cmH₂O to about 160 cmH₂O. This modification resulted in significant respiratory strengthening in normal subjects who utilized the modified device in the training program according to the subject invention. Still referring to FIG. 4, the valve of the Threshold Trainer, which is closed by the spring pressure, is originally perforated and has a latex flap to allow unidirectional airflow. This flap is subject to leakage and failure when used with higher spring constants producing pressure thresholds greater than 40 cmH₂O. Therefore, the device of the subject invention has been modified and improved by removing the latex flap and closing the perforations to create a solid valve 2. This also changed the manner in which the modified device is used. In order to take a breath according to the subject invention, the device must be removed from the subject's mouth and then replaced in the mouth for the breathing effort. The original Threshold Trainer also has a knob, which is used to turn the adjusting screw, with too small of a diameter. The modified device of the subject invention increased the size of the diameter of the knob 3 to a range of about 8-22 mm for an improved gripping and easier turning. The original Threshold Trainer is also used exclusively as a prescription device for patients under a physician's care. The modified device of the subject invention has been tested and proven effective for non-clinical uses, and thus the device is directed to non-clinical, over-the-counter distribution to all interested users desiring to strength train the respiratory muscles.

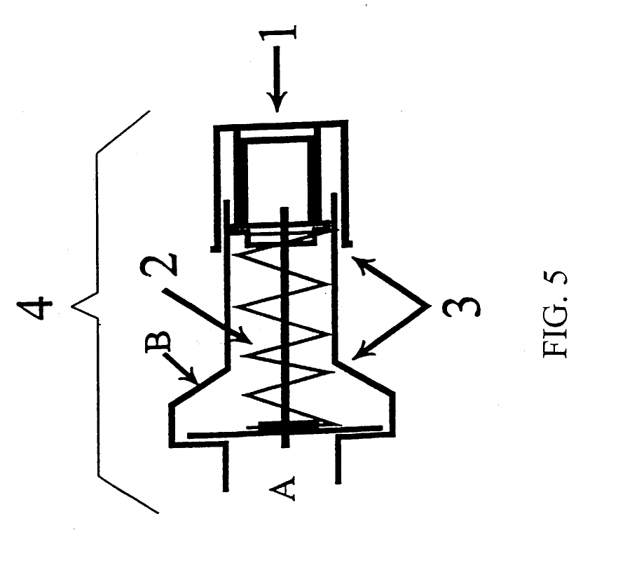

Referring now to FIG. 5, a modified positive end expiratory pressure (PEEP) valve device according to the subject invention is depicted. The valve changes the spring compression by a threaded cap 1 that pushes the sliding top spring retainer. The expired air goes through the mouthpiece A and out large openings on the side of the tube wall B. The spring constant provided by a conventional PEEP valve is too weak, generating a threshold pressure range of only 0-20 cmH₂O, to produce any meaningful expiratory muscle training in normal human subjects. Therefore, the modified device of the subject invention replaced the original spring with a spring 2 having a substantially elevated spring constant with a threshold pressure ranging from 0 cmH₂O to about 160 cmH₂O. In an embodiment, the threshold pressure is above about 50 cmH₂O. This modification results in significant expiratory muscle strengthening in normal subjects in experiments. The travel length for the threaded cap and thus the compression distance available for adjustment in a conventional PEEP valve is 1.5 cm. With such a short length, a quarter turn of the threaded cap produces an 8 cmH₂O change in threshold pressure. While this is marginally acceptable, the modified device of the subject invention has a 3 cm travel length 3 for a better adjustment of the spring compression for the usage of the device as a respiratory muscle trainer for normal human subjects.

In a preferred embodiment, the modified device is functionally adapted to generate a maximum expiratory pressure level ranging from 0 cmH₂O to about 160 cmH₂O. Preferably, the modified device provides a spring constant sufficient to increase the threshold expiratory pressure to a range of about 80 cmH₂O to 160 cmH₂O. With this modification, the device of the subject invention is available for non-clinical use on normal subjects desiring to increase their expiratory muscle strength. Utilizing this novel device as a tool, the present invention provides the Expiratory Muscle Trainer (EMT), a unique respiratory muscle training system, which is designed to train the muscles to work near their maximum force-generating capacity. This method of training provided by the present invention is based on a subject's ability to generate expiratory pressure being a function of the power generated by the expiratory muscles. Increasing expiratory muscle strength increases the pressure a subject can generate and increases the sound intensity of the subject's vocalization or even of a wind instrument played by the subject.

According to one embodiment of the subject invention, a person uses the modified expiratory threshold device for expiratory muscle training by breathing out (exhaling) through a mouthpiece attached to the expiratory end of the device. The device of the subject invention functions much the same way as the PEEP valve described above, in that the device's threshold pressure resists the subject's exhalation. However, the modified expiratory threshold device has a substantially heightened threshold pressure level, which requires the subject to generate stronger force from the expiratory muscles to sufficiently overcome the device's threshold pressure. When properly done, breathing through the device of the subject invention provides a maximum workout on these expiratory muscles, which may be equivalent to a person's workout of lifting weights at 75-80% of their maximum bodily strength.

In order to maximize the effect of the modified device in strengthening the respiratory muscles, the present invention also provides a non-clinical physical training program. Specifically, the training program is designed to strengthen the ability of the respiratory muscles to force air in and out of the lung. More specifically, the training program provides methods of breathing through the modified device to train the expiratory muscles to increase muscle strength by using daily exercise sessions comprised of short intense muscle exercises. For example, with the training program of the subject invention, the subject exercises the expiratory muscles for up to 30 minutes, preferably about 15-30 minutes, at least once, preferably twice, a day by breathing through the modified threshold device. The ultimate objective of the training program is to progressively increase the maximum expiratory pressure level a subject can generate, which results in an increased intensity of expiratory airflow in the subject's respiratory system.

In a preferred embodiment, the training program begins with measurement of the maximum expiratory pressure (MEP) a subject can normally generate. The modified threshold device is then adjusted to a threshold pressure which requires the respiratory muscles to generate forces at about 75-80% of the MEP. This means that the subject must produce an isometric force up to about 75-80% of MEP before the valve opens. Preferably, the training is carried with the modified device's opening pressure set at about 75% of the subject's MEP in daily sessions. With higher settings, the subject experiences a greater effort to maintain the threshold pressure, which provides an exercising effect on the expiratory muscles. Following this daily exercise schedule of using the modified expiratory threshold device and training at an expiratory pressure of about 75% maximum expiratory pressure the subject should experience substantial increases in maximum expiratory pressures within 4 weeks of training.

In yet another preferred embodiment, the training program provides a strengthening protocol consisting of a high-load expiratory muscle training designed to improve maximal expiratory force production by utilizing the modified device. The device of the subject invention provides an adjustable threshold load to expiration, and the load is regulated by adjustment of the spring compression. In one embodiment, the training program provides daily training sessions to be conducted about 5 times a week. Each daily training session, can, for example, consist of about four sets of six training breaths for a total of 24 training breaths. A subject blows through the device at about 75%-80% of the subject's initial maximum expiratory pressure (MEP). A training breath preferably last roughly 2-4 seconds, separated by a 10 to 15 second rest. The daily training lasts approximately 15-20 minutes. At the end of week one, the new MEP for the subject is measured and the threshold pressure is adjusted to about 75%-80% of the new MEP. The training is repeated on a daily basis over three to four weeks.

In a preferred embodiment, a subject expires through the device at a load of about 80 cmH₂O threshold pressure. More preferably, subject expires through the device at a load of about 100 cmH₂O threshold pressure. Even more preferably, a subject expires through the device at a load of about 120 cmH₂O threshold pressure. Yet in a more preferred embodiment, a subject expires through the device at a maximum load of about 160 cmH₂O threshold pressure.

In an alternative embodiment, the modified expiratory threshold device of the subject invention is used for treating patients with vocal disorders. Specifically, the threshold device of the subject invention is adjusted to require the expiratory muscles to generate expiratory forces at about 75-80% of the maximum expiratory pressure (MEP) the person can generate. This means that the patient must produce an isometric force up to about 75-80% of MEP before the valve opens. The patient must maintain that force level to produce expiratory airflow. Expiring at this force level for 2-3 seconds exercises the expiratory muscles in a manner that requires expiratory muscle work. This work-out is called expiratory muscle strength training, and this work-out maybe broken into many shorter time-intervals of exercise to total up to a daily exercise ranging from 15 to 30 minutes according to the individual patient's preferences and needs.

In an alternative embodiment, the modified expiratory threshold device of the subject invention is used for treating patients with COPD. Specifically, the threshold device of the subject invention is adjusted to require the expiratory muscles to generate expiratory forces at about 75-80% of the maximum expiratory pressure (MEP) the person can generate. This means that the patient must produce an isometric force up to about 75-80% of MEP before the valve opens. The patient must maintain that force level to produce expiratory airflow. Expiring at this force level for 2-3 seconds exercises the expiratory muscles in a manner that requires expiratory muscle work. This work-out is called expiratory muscle strength training, and this work-out maybe broken into many shorter time-intervals of exercise to total up to a daily exercise ranging from 15 to 30 minutes according to the individual patient's preferences and needs.

The expiratory muscle strength training of the subject invention is very different from the more common, low pressure endurance expiratory training methods at pressures of only 10-50% MEP or any other expiratory therapy treatments applied to these patients. The conventional, low pressure expiratory endurance training produces little or no increase in maximum expiratory pressure and thus no therapeutic value, whereas the training devices and methods according to the subject invention effectively provides substantial (35-100% or more) increases in maximum expiratory pressure in less than four weeks of training.

As the person increases their expiratory muscle strength, the expiratory threshold valve is adjusted to keep the training pressure at about 75-80% of the elevated maximum expiratory pressure (MEP) up to a training pressure of about 160 cmH₂O. After the person reaches 160 cmH₂O, further increases in expiratory muscle strength can be achieved by increasing the number of breathing efforts for each training session. After an acceptable plateau of expiratory muscle strength is reached, sustained training will keep the expiratory muscles stronger. As a person's expiratory muscles increase in strength, he or she will be able to adjust the expiratory threshold valve and the training schedule to achieve further increases in maximum expiratory pressure.

The users can get increases in expiratory muscle strength by increasing the expiratory training pressure or increasing the number of expiratory efforts (repetitions). This training method according to the subject invention will be useful to all persons interested in increasing maximum expiratory pressure such as athletes, singers, speakers and speech pathologists requiring them to produce the sound with expiratory airflow. The use of this method is also recommended with aging adults that experience a diminished voicing ability and for patients suffering COPD. The benefits of this training are an increased intensity of expiratory airflow and/or an increased ability to sustain an expiratory airflow magnitude. Accordingly, the training system of the subject invention will not only help patients with vocal disorders, but aging adults, healthy vocalists, band students, athletes, and the population of public speakers.

The subject invention provides high intensity and low repetition expiratory specific exercises to significantly increase expiratory pressure generating capacity in subjects. The subject invention provides substantial muscle training effect. This simple method of expiratory specific strength training is not only effective but also efficient for increasing expiratory pressure support in subjects.

While the contemplated uses of the device and methods of the subject invention are exemplified herein by uses on human subjects, the subjection invention can be fitted for animal use in training the respiratory muscles of animals.

It should be understood that the examples and embodiments described herein are for illustrative purposes only and that various modifications or changes in light thereof will be suggested to persons skilled in the art and are to be included within the spirit and purview of this application and claims.

We claim:

1. A method for conditioning respiratory muscles in a patient wherein said method comprises using a pressure relief valve comprising a spring constant capable of having a pressure threshold above about 50 cmH₂O; and wherein said method comprises positioning said pressure relief valve in the patient's mouth during exhalation of at least one breathing cycle, such that said pressure threshold resists the patient's exhalation until sufficient force is produced to overcome the pressure threshold.

2. The method for conditioning respiratory muscles according to claim 1, wherein said pressure threshold is set between about 80 cmH₂O and 160 cmH₂O.

3. The method for conditioning respiratory muscles according to claim 2, wherein said pressure threshold is set between about 120 cmH₂O and 160 cmH₂O.

4. The method for conditioning respiratory muscles according to claim 1, wherein said pressure threshold is about 160 cmH₂O.

5. The method for conditioning respiratory muscles according to claim 1, further comprising the step of measuring a maximum expiratory pressure generated by the patient.

6. The method for conditioning respiratory muscles according to claim 5, wherein the force produced to overcome the pressure threshold is at least about 75% of the maximum expiratory pressure.

7. The method for conditioning respiratory muscles according to claim 5, wherein the force produced to overcome the pressure threshold is about 75% of the maximum expiratory pressure.

8. The method for conditioning respiratory muscles according to claim 1, wherein the patient uses the pressure valve throughout a plurality of breathing cycles.

9. The method for conditioning respiratory muscles according to claim 8, wherein the duration of the plurality of breathing cycles is up to about 30 minutes, performed at least once a day.

10. The method for conditioning respiratory muscles according to claim 9, wherein the plurality of breathing cycles are performed twice a day.

11. The method for conditioning respiratory muscles according to claim 8, wherein the duration of the plurality of breathing cycles is about 5 minutes to 30 minutes, at least once a day.

12. The method for conditioning respiratory muscles according to claim 1, wherein the breathing cycles comprises about 25 exhalations.

13. A pressure relief valve device for conditioning respiratory muscles in a patient comprising:
a mouthpiece;
at least one threshold pressure valve; and
a spring in contact with the valve, wherein the spring produces a spring constant capable of having a threshold pressure above about 50 cmH₂O to open the valve.

14. The device according to claim 13, further comprising an adjusting screw and a knob to turn the adjusting screw.

15. The device according to claim 13, further comprising a sliding top spring retainer and a threaded cap.

16. The device according to claim 15, wherein the threaded cap has at least about a 3 cm travel length.

17. A method for treating a patient having obstructive sleep apnea syndrome comprising:
measuring the maximum expiratory pressure that the patient normally generates during exhalation;
selecting a pressure relief valve comprising a pressure threshold above about 50 cmH₂O;
adjusting the pressure threshold such that when the patient uses the pressure valve, the pressure threshold resists the patient's exhalation until the patient produces at least about 75% of the patient's maximum expiratory pressure to overcome the pressure threshold; and
positioning the pressure relief valve in the patient's mouth during exhalation of at least one breathing cycle.

18. The method according to claim 17, wherein the pressure threshold is adjusted so that 75% of the patient's maximum expiratory pressure overcomes the pressure threshold.

19. The method according to claim 17, wherein the patient uses the pressure relief valve throughout a plurality of breathing cycles.

20. The method according to claim 19, wherein the duration of the plurality of breathing cycles is up to about 30 minutes.

21. The method according to claim 17, wherein the breathing cycles are performed at least once a day.

22. The method according to claim 17, wherein the breathing cycles are performed about five times a week.

23. A method for treating a patient diagnosed with a vocal disorder comprising:
measuring the maximum expiratory pressure that the patient normally generates;
selecting a pressure relief valve comprising a pressure threshold above about 50 cmH₂O;
adjusting the pressure threshold such that when the patient uses the pressure valve, the pressure threshold resists the patient's exhalation until the patient produces at least about 75% of the patient's maximum expiratory pressure to overcome the pressure threshold; and
positioning the pressure relief valve in the patient's mouth during exhalation of at least one breathing cycle.

24. The method according to claim 23, wherein the pressure threshold is adjusted so that 75% of the patient's maximum expiratory pressure overcomes the pressure threshold.

25. The method according to claim 23, wherein the patient uses the pressure relief valve throughout a plurality of breathing cycles.

26. The method according to claim 25, wherein the duration of the plurality of breathing cycles is up to 30 minutes.

27. The method according to claim 23, wherein the breathing cycles are performed at least once a day.

28. The method according to claim 23, wherein the breathing cycles are performed about five times a week.

29. A method for treating a patient diagnosed with a chronic obstructive airways disease comprising:
measuring the maximum expiratory pressure that the patient normally generates;
selecting a pressure relief valve comprising a pressure threshold above about 50 cmH₂O;
adjusting the pressure threshold such that when the patient uses the pressure valve, the pressure threshold resists the patient's exhalation until the patient produces at least about 75% of the patient's maximum expiratory pressure to overcome the pressure threshold; and
positioning the pressure relief valve in the patient's mouth during exhalation of at least one breathing cycle.

30. The method according to claim 29, wherein the pressure threshold is adjusted so that 75% of the patient's maximum expiratory pressure overcomes the pressure threshold.

31. The method according to claim 29, wherein the patient uses the pressure relief valve throughout a plurality of breathing cycles.

32. The method according to claim 31, wherein the duration of the plurality of breathing cycles is up to about 30 minutes.

33. The method according to claim 29, wherein the breathing cycles are performed at least once a day.

34. The method according to claim 29, wherein the breathing cycles are performed about five times a week.
